(19) United States
(12) Patent Application Publication
(10) Pub. No.: US 2005/0085836 A1
(43) Pub. Date: Apr. 21, 2005

(54) Methods and devices for endothelial denudation to prevent recanalization after embolization

(76) Inventor: Jean Raymond, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/938,662

(22) Filed: Sep. 13, 2004

Related U.S. Application Data
(60) Provisional application No. 60/502,255, filed on Sep. 12, 2003.

(51) Int. Cl. A61B 17/22

(52) U.S. Cl. 606/159

(57) ABSTRACT

A device for denuding the endothelium of a vascular cavity wall in order to maximize thrombosis and prevent recanalization in the vascular cavity. The device comprises an endovascular tool, such as a guidewire, microcatheter, microballoon or coil, and at least one denuding element attached to the endovascular tool, adapted to remove at least part of the endothelium of the vascular cavity wall while avoiding wall perforation. This invention also includes a method of preparing a vascular cavity having blood therein before vascular cavity occlusion by removing at least part of the endothelium of the cavity wall in order to maximize thrombosis and prevent recanalization in the vascular cavity. The method comprises the steps of endovascularly disposing at least one denuding element inside the cavity and using the at least one denuding element to remove at least part of an endothelium of a cavity wall while avoiding wall perforation.

### BACKGROUND OF THE INVENTION

1. Field of the Invention

The present invention relates to endovascular treatment of vascular diseases. More particularly, the present invention relates to a method and device to prevent recanalization after vascular occlusion procedures.

2. Background Art

Intracranial aneurysms are small saccular dilatations that arise at bifurcations on arteries located in the subarachnoid spaces at the base of the brain. They are present in 1 to 5% of the adult population, but remain asymptomatic until the day they rupture, causing a cerebral sub-arachnoid hemorrhage (SAH), an event that occurs in 28,000 patients a year in North America. SAH is associated with a significant morbidity and mortality despite modern medical and surgical management.

Surgical treatment of aneurysms is increasingly being replaced by a less invasive “endovascular” alternative. This new treatment uses a femoral approach to reach and occlude aneurysms by packing the cavity with detachable platinum coils, as shown for example in U.S. Pat. No. 5,540,680 issued on Jul. 30, 1996, to Guglielmi et al. The main advantage of the method remains its safety: a very soft platinum coil is introduced from the femoral artery into the aneurysm, with more ease and less risk of aneurysm rupture than with detachable balloons. The coil may be repositioned to the satisfaction of the operator as it remains attached to the coil pusher until the desired moment of detachment. The aneurysm is progressively packed as densely as possible with a number of platinum coils, ideally until the lesion is completely excluded.

This endovascular treatment is successful in preventing re-bleeding in the acute phase and it is now the preferred method of treatment in many centers for patients with aneurysms that have a higher surgical risk. The endovascular approach can improve the outcome of patients suffering a SAH from aneurysmal rupture as compared to surgical clipping. While treatment in the acute phase after rupture is imperative to prevent re-bleeding, the management of unruptured aneurysms remains controversial because of a low risk of hemorrhage and a high surgical risk.

Unruptured aneurysms are increasingly being discovered incidentally during cerebral imaging performed for unrelated symptoms. An effective endovascular treatment may offer a less morbid alternative to surgical treatment of unruptured aneurysms and thus prevent the morbidity associated with SAH. Unfortunately, the current endovascular treatment implies an often incomplete occlusion near the aneurysm neck (i.e. the junction between the sac of the lesion and the parent vessel) especially in wide-necked aneurysms, in order to prevent stenosis or clot formation inside the normal artery. Endovascular treatment is thus frequently incomplete and leads to early recurrence in at least 20% of patients, necessitating ineffective and more risky retreatments. This drawback is the main reason why most patients are still being treated with surgical clipping after craniotomy. A significant improvement of the long-term efficacy is essential if we want this minimally invasive strategy to be offered to more patients currently being treated with conventional surgery.

It has been shown that recanalization of aneurysms after endovascular treatment can be inhibited by in situ beta radiation using radioactive coils. These mechanisms are still poorly understood, but relate to inhibition of endothelial invasion of the clot that forms after coil occlusion. However, radioactive stents in canine lateral wall aneurysms have been used to show that persisting flow at the level of residual necks after treatment cannot be occluded by radiation and that it only seems to prevent recanalization once there is thrombus formation. Furthermore, the effective target activities are difficult to reach in large, wide-necked aneurysms, since such aneurysms have a tendency to be incompletely occluded after treatment. See Raymond et al., “In Situ beta radiation to prevent recanalization after coil embolization of cerebral aneurysm”, Stroke, February 2002, pp. 421-427, “Beta radiation and inhibition of recanalization after coil embolization of canine arteries and experimental aneurysms: How should radiation be delivered?”, Stroke, May 2003, pp. 1262-1268, and “Long-term angiographic recurrences after selective endovascular treatment of aneurysms with detachable coils”, Stroke, June 2003, pp. 1398-1403.

Although the exact mechanisms involved in recurrences after coil embolization remain to be determined, Raymond et al. have established in “Role of endothelial lining in persistence of residual lesions and growth of recurrences after endovascular treatment of experimental aneurysms”, Stroke, March 2002, pp. 850-855, the value of endothelial denudation in decreasing residual necks and recurrences after sponge embolization of canine aneurysms, a model that has shown a strong propensity for early recurrences. In this publication, it was established that the endothelial lining is responsible for persistence of residual lesions and that early endothelial invasion of the clot leads to recanalization and recurrences after embolization of aneurysms. The hypothesis was that the endothelium lining residual necks after endovascular treatment prevented thrombosis, and subsequent neointimal cell colonization and healing, at the interface between the embolic agent and the aneurysmal wall. In an experiment where the endothelial covering of the venous pouch was mechanically removed before aneurysm construction and sponge embolization, there was extensive fibrous tissue replacement of the space not occluded by the sponge, at the neck. Although not completely eliminated, the formation of clefts, particularly at the interface between the sponge and the aneurysmal wall, was strikingly diminished. It was therefore established that a strategy aiming at removing the endothelial layer before deposition of radioactive coils could promote thrombosis of residual necks, decrease endothelial invasion and recanalization of the clot and improve long-term results of embolization.

Accordingly, there is a need for appropriate tools to accomplish an effective denudation of the intima of the vessel to be occluded, in an effort to prevent recanalization, and thus improve the long-term efficacy of endovascular treatment.

### SUMMARY OF INVENTION

It is therefore an aim of the present invention to provide a device for effective denudation of the intima of a vessel to be occluded in order to prevent recanalization.

It is another aim of the present invention to provide a device which can be incorporated onto conventional tools used for endovascular treatment of an aneurysm.

Therefore, in accordance with the present invention, there is provided a device for denuding the endothelium of a vascular cavity wall, the device comprising an endovascular tool for performing an endovascular treatment of the vascular cavity, and at least one denuding element adapted to remove at least part of the endothelium of the vascular cavity wall while avoiding wall perforation, the at least one denuding element being attached to the endovascular tool, whereby the denuding of the endothelium of the vascular cavity wall maximizes thrombosis and prevents recanalization in the vascular cavity.

Further in accordance with the present invention, there is also provided a method of preparing a vascular cavity having blood therein before vascular cavity occlusion, the method comprising the steps of endovascularly disposing a denuding element inside the cavity, and using the denuding element to remove at least part of an endothelium of a cavity wall while avoiding wall perforation, whereby the removal of at least part of the endothelium of the cavity wall maximizes thrombosis and prevents recanalization in the vascular cavity after vascular cavity occlusion.

All references cited are incorporated herein by reference.

### BRIEF DESCRIPTION OF THE DRAWINGS

Having thus generally described the nature of the invention, reference will now be made to the accompanying drawings, showing by way of illustration a preferred embodiment thereof and in which:

FIG. 1 is a schematic longitudinal cross-sectional view of a treatment of an aneurysm according to the prior art;

FIG. 2 is a schematic perspective view of a denudation device on a guidewire according to a preferred embodiment of the present invention;

FIGS. 3A-M are schematic side views of denudation devices on a guidewire according to alternative embodiments of the present invention;

FIGS. 4A-B are schematic side views of the denudation device on a microcatheter according to another embodiment of the present invention;

FIGS. 5A-B are schematic side views of the denudation device on coils according to yet another embodiment of the present invention;

FIG. 6 is a schematic longitudinal cross-sectional view of a treatment of an aneurysm using the denudation device according to the embodiment of FIG. 2;

FIG. 7 illustrates endovascular denudation and effects on recanalization. Macroscopic stereophotographs of “en face” views of denuded lumens immediately after treatment (a, b), and of cross-sectioned arteries 3 months after treatment by denudation only (d), coil embolization only (e), or endothelial denudation followed by coil embolization (f), or left intact (c). The device sometimes caused superficial lacerations of the intima (b);

FIG. 8 illustrates recanalization after coil occlusion. Hispathological findings 10 days (a) and 12 weeks (b) after coil embolization;

FIG. 9 illustrates absence of recanalization after denudation and coil embolization. Hispatological finding 12 weeks after mechanical denudation followed by coil embolization showing complete filing of vascular lumen with vascularized connective tissue. Movat pentachrome stain (×20 (a) or ×200 (b)); and

FIG. 10 illustrates angiographic and macroscopic results after denudation and coiling of bifurcation aneurysms. Selected views from angiographic studies performed immediately (a, d) and 3 months (b, e) after coiling, preceded (d, e) or not (a, b) by endothelial denudation.

### DESCRIPTION OF THE PREFERRED EMBODIMENTS

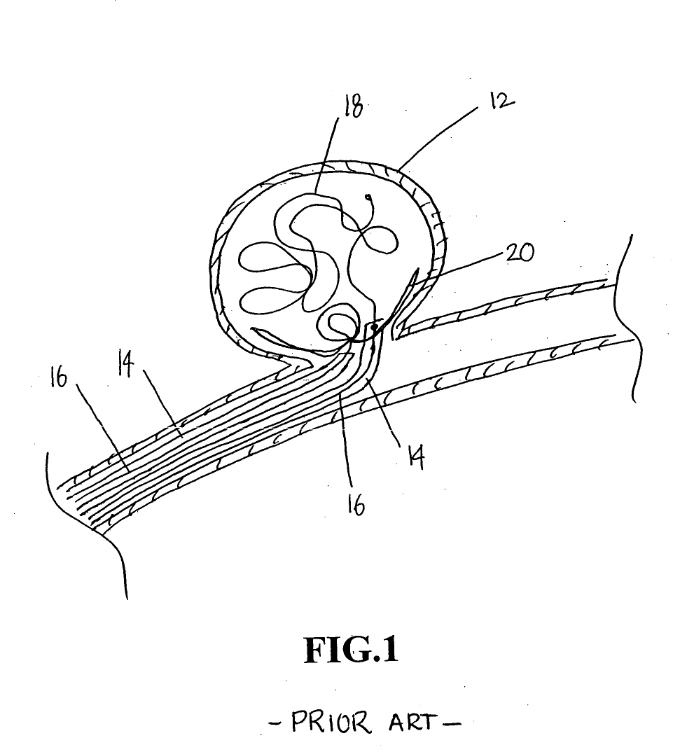

Referring to FIG. 1, the basic tools necessary for endovascular treatment of an aneurysm 12 include the following: a microcatheter 14, which is a small caliber empty tube that permits the introduction of wires and devices to the site of the vascular lesion, a guidewire 16, which is a usually a torquable filament that is used to guide the microcatheter 14 to the lesion site, and an embolic agent 18, which is most frequently a platinum coil but could also be a polymer compound or any other occlusive substance. Other endovascular tools include microballoons, detachable or not, stents, membranes, sponges, and special tools such as an aneurysmal neck bridge device 20, one example of which is presented in U.S. Pat. No. 5,935,148, issued on Aug. 10, 1999 to Villar et al.

The denudation device 10 of the present invention is designed to mechanically, physically or chemically denude the endothelial layer of the aneurysm wall using a denuding element attached to one of these basic endovascular tools. Just as multiple types of blades, clips, forceps, curettes, retractors, sponges, etc. are necessary to accomplish a surgical task, the present invention is meant to provide the operator with a variety of tools that will permit endothelial denudation of the vessel, or of the neck of aneurysms.

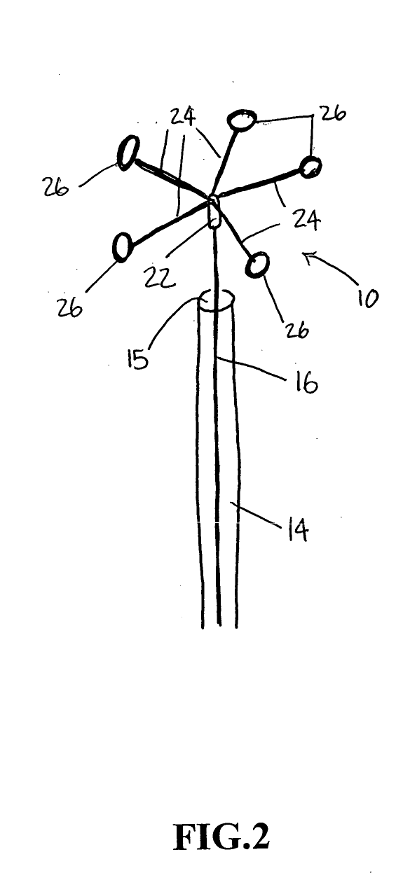

Referring to FIG. 2, a preferred embodiment of the denudation device 10 is a transcatheter torquable device made of nitinol. The denudation device 10 is detachably mounted on a tip of the guidewire 16 and comprises a stem 22 supporting radiating strands 24. The strands 24 are soft in order to prevent perforations, and can be oriented radially from the stem. Ideally, each strand 24 ends with a small loop 26 made of nitinol or polymeric suture material. The denudation device 10 is sized so as to be able to travel through the microcatheter 14 and exit through a distal wire hole 15 thereof.

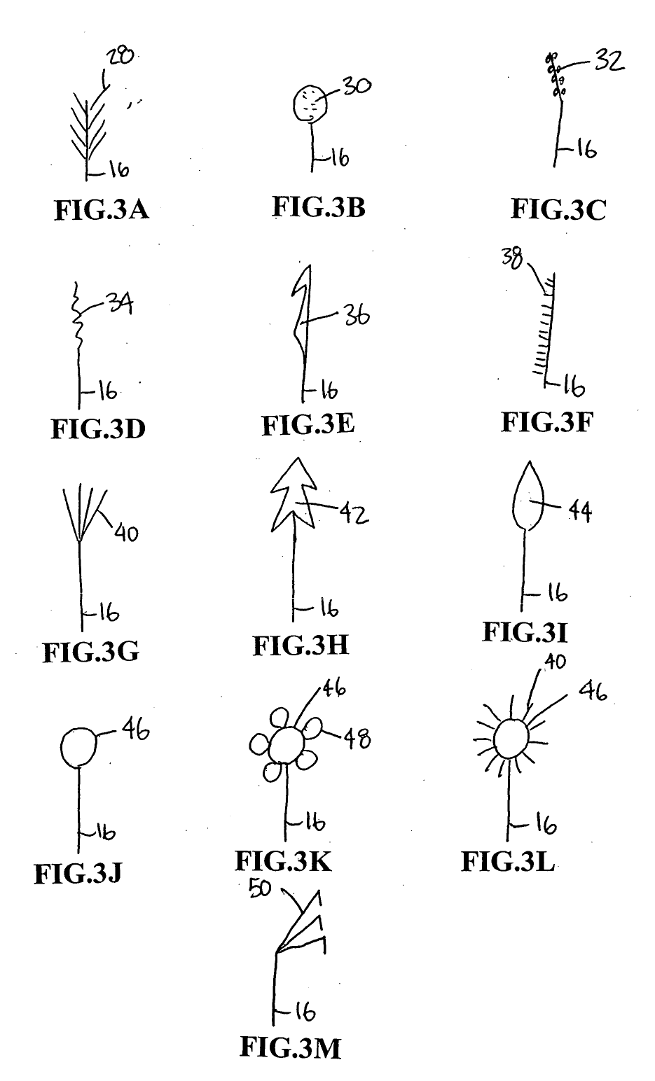

Referring to FIGS. 3A-M, the denudation device 10 mounted on the tip of the guidewire 16 can also be of various other configurations, for example with: bristles 28 (FIG. 3A), a sponge 30 (FIG. 3B), a sandy surface 32 (FIG. 3C), an irregular wire tip 34. (FIG. 3D), a cutting tip 36 (FIG. 3E), a brush 38 (FIG. 3F), filaments 40 (FIG. 3G), a harpoon-like tip 42 (FIG. 3H), a curette-like tip 44 (FIG. 3I), a loop 46 (FIG. 3J), a loop 46 and daughter loops 48 (FIG. 3K), a loop and filaments 40, (FIG. 3L), a rake-like tip 50 (FIG. 3M), or any other appropriate shape or combination of shapes. However, a denudation device 10 with single strands is preferred to a lasso-like type in order to prevent the catching of coils during de-endothelialization. The denudation device 10 can also take the form of a membrane, with or without reinforcements, that can be made of appropriate polymers, metals or biodegradable materials.

It is also considered to use a microcatheter with an additional sidehole used as an exit port for the denudation device 10 and guidewire 16. This configuration will decrease the risk of fundus perforation by directing the tool toward the side walls. The denudation device 10 can also be larger than the distal wire hole 15 of the microcatheter 14 and be located at the extremity thereof with the guidewire 16 inserted in the microcatheter 14. As an alternative, the guidewire 16 with a denudation device 10 could be externally attached to the microcatheter 14.

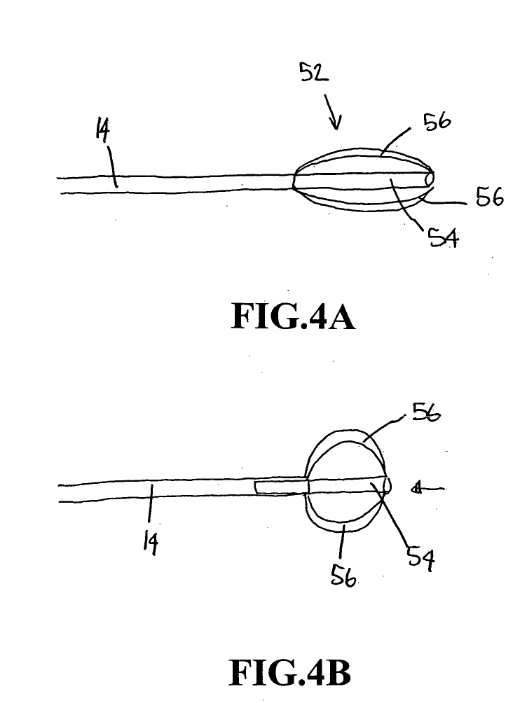

In another embodiment of the invention, the denudation device is mounted on a tip of the microcatheter 14. In this case, the microcatheter 14 and denudation device are preferably made of nitinol, but another appropriate material can be used. The denudation device of this embodiment can be of various configurations, similar to the denudation devices of FIGS. 3A-M described above. Referring to FIGS. 4A-B, another type of denudation device tip considered is an egg-beater-like tip 52, with a retractable portion 54 that extends side loops 56 when retracted. Such a design presents the advantage of protecting the aneurysm fundus from perforation by the denudation device, orienting the denuding element (i.e. side loops 56) toward the side walls.

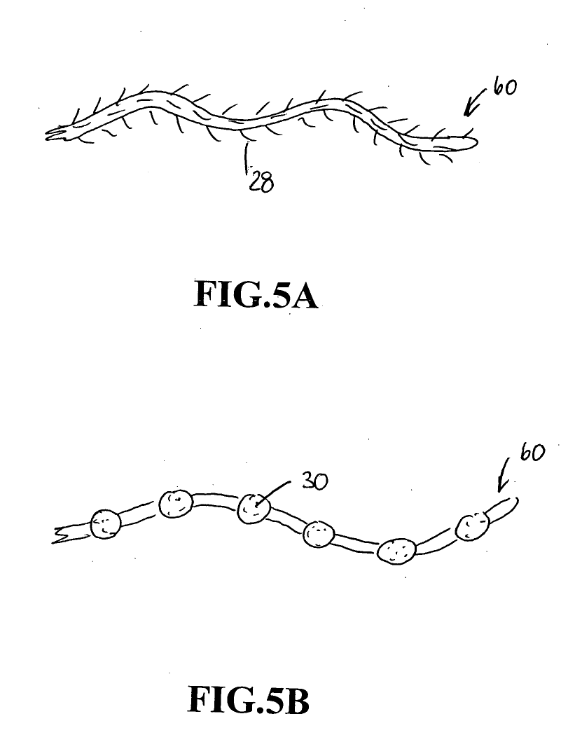

Referring to FIGS. 5A-B, a further embodiment of the invention is a modified coil 60 used as a denudation device. In this case, the goal is to scrape the endothelium as the modified coil 60 is deployed near the neck of the aneurysm. The modified coil 60 can be made of an appropriate metal or polymer. The modified coil 60 is structurally similar to a standard coil 18, but the modified coil surface is made so as to be able to strip the endothelium. This is done preferably with bristles 28 (FIG. 5A) made of nitinol or any other appropriate material, but a variety of other scraping surfaces can be used, for example sponges 30 (FIG. 5B), microcutting edges, scales, sand or salt coating.

It is also considered to include the denudation device on other embolic devices, such as specially designed microballoons or sponges with a scraping surface including, for example, bristles, scales, or sand, similar to the modified coil surface previously described.

Alternatively, it is considered to use a denudation device damaging the endothelial layer by using chemical means, for example a coil, balloon, sponge, catheter or any other appropriate endovascular tool coated with or otherwise delivering a chemical agent. Appropriate chemical agents include, for example, sclerosing agents toxic to the endothelium such as Sotradecol, hypertonic saline, ethanol, or calcium chloride; cytotoxic agents such as Tetracycline; or inhibitors of smooth muscle cells such as Tamoxifen, Sirolimus, Actinumycin, or 27OH Cholesterol. These toxic chemical agents could be in the form of gels, creams, pastes, ointments, or any other appropriate form.

It is also considered to provide a denudation device damaging the endothelial layer by using physically toxic means. For example, heat or cold could be applied through a microballoon to damage the endothelium. Also, electricity or radiofrequency could be used to damage the endothelial layer by using one or more electrodes linked to an electrical or radiofrequency power source as a denudation device attached to an endovascular tool. The denudation device could also use laser to effectively remove the endothelial layer, for example by using an optical fiber delivering laser energy as a denudation device.

It is understood that the denudation device used must be able to damage the endothelium while avoiding wall perforation and damage to the parent vessel and surrounding tissue. Denudation devices using other appropriate mechanical, chemical or physical means to damage the endothelium will be readily apparent to those skilled in the art. These variations are intended to be included within the scope of this invention.

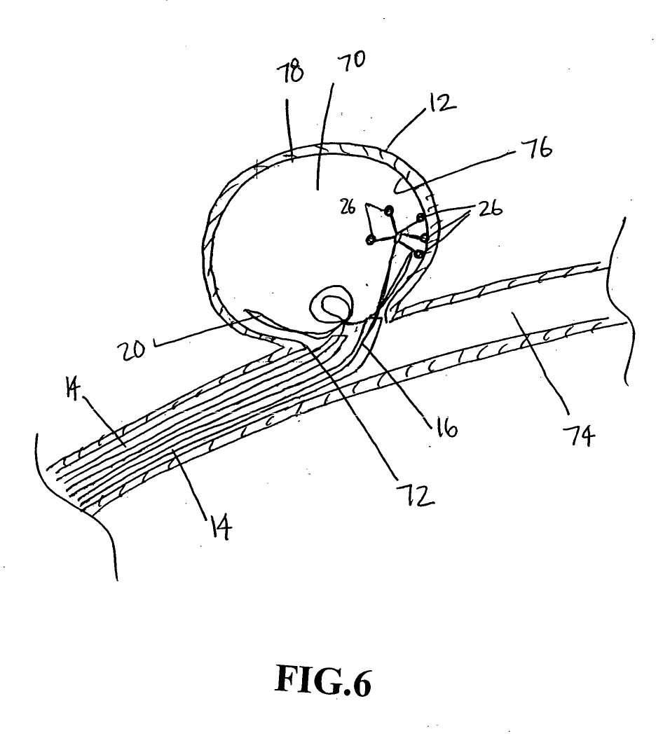

Referring to FIG. 6, an example of the use of the denudation device 10 of the preferred embodiment of FIG. 2 to maximize thrombosis in a aneurysm cavity 70 will be given. Preferably, a microcatheter 14 is first guided near a neck 72 of the aneurysm cavity 70 by going through a parent vessel 74 in the manner already known in the art. An aneurysmal neck bridge device 20 is then preferably placed at the neck 72 of the aneurysm cavity 70 through the lumen of the microcatheter 14 in the manner known in the art in order to protect the parent vessel 74 from accidental scraping. The denudation device 10 and guidewire 16 supporting it are delivered to the aneurysm cavity 70 through the lumen of the same or a second microcatheter 14. The loops 26 of the denudation device 10 are gently scraped against a cavity wall 76, especially at the neck 72, in order to remove the endothelium thereof. Care must be taken to avoid wall perforation, especially at a fundus 78 of the aneurysm cavity 70, and scraping or emboli of the parent vessel 74. In order to protect the fundus 78, the aneurysm cavity 70 can be partially filled with coils (not shown) before introducing and using the denudation device 10. Depending on the aneurysm size and shape, a variety of other denudation devices such as previously described can be used successively in order to optimize endothelium removal. In the case of a detachable denudation device 10, the denudation device 10 can be detached and left in the aneurysm cavity 70 after scraping in order to act as an additional occlusion material in a way similar to standard coils. After the scraping of the cavity wall 76 is satisfactory, the aneurysm cavity 70 is occluded, preferably with coils (not shown), in order to promote thrombosis.

The following Experiments A and B serve to better understand the present invention. It is to be understood that these examples in no way serve to limit the true scope of the invention, but rather are presented here for illustrative purposes.

### Experiment A

It has been previously shown in animal models that coil embolization is routinely followed by recanalization.

Experiments were performed using the single coil arterial occlusion model (described in Raymond et al., “In Situ beta radiation to prevent recanalization after coil embolization of cerebral aneurysm”, Stroke, February 2002, pp. 421-427), but preceded by endothelial denudation performed by a transcatheter technique using the preferred embodiment of the denudation device 10 shown in FIG. 2. Typically 5 passes with rotations were performed in each arterial segment. The group of arteries studied included 16 arteries that were denuded with the denudation device 10 and coiled, 6 arteries that were coiled without de-endothelialization, and 6 arteries that were denuded with the denudation device 10 but not coiled. Six (6) control arteries were also studied for comparison. Follow-up angiography was performed immediately after embolization, at 1 hour, 4 and 12 weeks, immediately before sacrifice. In 2 animals arteries were studied by pathology at 10 days. All arteries embolized with platinum coils were initially occluded but recanalized while most arteries (12/16 or 80%) denuded before coil embolization remained occluded at 3 and 12 weeks. Arteries that were denuded but not coiled were angiographically intact for the entire duration of the experiment. At post-mortem photography, arteries treated with coils only were recanalized while most arteries treated by de-endothelialization an coiling were occluded with conjunctive tissue filling the lumen.

Denudation of the lining of canine T-type bifurcation aneurysms using the denudation device 10 shown in Fig. was then performed in 7 animals and angiographic and pathological results compared to 7 control aneurysms treated with coils only. There were no thromboembolic complications, except in one preliminary animal in which the parent artery was also denuded. This model has previously shown that it could detect embolic complications from delayed migration of polymeric agents or clots. See Raymond et al., “Alginate for endovascular treatment of aneurysms and local growth factor delivery”, American Journal of Neuroradiology, June/July 2003, pp. 1214-1221. Angiographic scores were significantly improved at 12 weeks in the group treated by denudation and coiling as compared to coiling only (p=0.011; Wilcoxon-Mann-Whitney test). Neointimal scores were also significantly better (p=0.03; Wilcoxon-Mann-Whitney test).

### Experiment B

The goal of that experiment was to study the effects of mechanically disrupting the endothelial layer with an endovascular device immediately before coil deposition, first in canine arteries, then in bifurcation aneurysms, in an effort to prevent recanalization and recurrences after coil embolization.

Material and Methods

Animal Models

Protocols were approved by the Institutional Animal Care Committee in accordance with guidelines of the Canadian Council of Animal Care. All procedures were performed under general anesthesia. Twenty-seven beagles weighing 10 to 15 kg were sedated with acepromazine (0.1 mg/kg), glycopyrrolate (0.01 mg/kg), and butorphanol (0.1 mg/kg), and anesthetized with intravenous thiopental (15 mg/kg). Animals were ventilated artificially and maintained under anesthesia with 2% isoflurane. Postoperative analgesia was provided for 3 days by a 50-μg Fentanyl skin patch.

Arterial Occlusion Model

The arterial occlusion model has previously been described (Raymond J. et al, Stroke 2002; 33:421-427; Raymond J.et al, Stroke 2003; 34:1262-1268). Briefly, a percutaneous femoral puncture was used to reach bilateral maxillary or vertebral arteries with 2-French microcatheters (Excelsior; Target Therapeutics). It has been previously shown that single-coils occlusion of vertebral or maxillary arteries routinely leads to thrombosis by 1 hour, followed by recanalization by 2 weeks (Raymond J. et al, Stroke 2002; 33:421-427; Raymond J.et al, Stroke 2003; 34:1262-1268).

Angiographic and pathological findings are identical, whether arteries are maxillary or vertebral. A 3-mm-caliber 0.015 platinum coil 8 cm in length (Target Therapeutics) was implanted into a 10-mm segment of each artery, with or without de-endothelialization. Denudation of the arterial wall was accomplished using an aneurismal neck-bridge device (AND). This platinum-covered nitinol device was designed to assist coiling- of wide-necked aneurysms. It is composed of 3 loops 3 mm in diameter attached by a common stem. Endothelial denudation had been verified in 6 preliminary animals by en face photography of autopsy specimens stained with Evans blue as well as by sections of normal or denuded arterial segments fixed immediately for pathological confirmation that the endothelium was intact or denuded. Typically, 5 passes with rotations were performed in each arterial segment. We studied 16 arteries that were denuded and coiled, whereas 16 arteries were coiled without denudation, and 6 were denuded with the AND but not coiled. Six intact arteries served as controls. Angiography was performed immediately after embolization, at 1 hour, 4 weeks, and 12 weeks, immediately before euthanization. In 2 animals arteries were studied by angiography and pathology at 10 days. Multiple projections after selective injections were interpreted in a blind fashion. Occlusion was defined as the absence of antegrade blood flow through the arterial segment. Any antegrade contrast opacification was sufficient to label the artery recanalized.

Bifurcation Aneurysm Model

In 1 animal, the aneurysm was constructed on a Y-type bifurcation, constructed as described. (Raymond J. et al, Radiology 2001; 221:318-326; Raymond et al., AJNR AM J Neuroradiol. 2003; 24:1778-1784) Terminal bifurcation aneurysms were constructed in 15 animals after a T-type bifurcation was created between the 2 common carotid arteries as described. (Raymond J. et al, Stroke 2002; 33:421-427; Raymond J. et al, AJNR Am J Neuroradio. 2002; 23:129-138) Bifurcation aneurysms were measured by angiography immediately before the endovascular procedure. Embolization was performed exactly as described in 7 control animals. (Raymond J. et al, AJNR Am J Neuroradiol. 2002; 23:1710-1716; Raymond J. et al, AJNR Am J Neuroradiol. 2002; 23:129-138) Briefly, after a first coil of a diameter approaching the size of the aneurysm was detached, coils of decreasing diameters were introduced to pack the lesion until complete or near-complete obliteration. In 8 aneurysms submitted to denudation before coil embolization, a 12- or 16-mm AND was first introduced and rotated at the level of the neck of the lesion during 1 minute before retrieval. Coiling was then performed as described, but the total length of coils introduced was voluntarily decreased as compared with controls, to prevent thrombotic complications, because clot formation and aneurismal sac occlusion tended to occur earlier during coil introduction. Transfemoral angiography was undertaken immediately after embolization, at 3 weeks, and at 3 months. Results were scored according to a previously described classification. (Raymond J. et al., AJNR Am J Neuroradiol. 2002; 23:129-138) A score of 0 indicated complete obliteration; 1, minimal residual or recurring neck (“dog ears”); 2, a more sizable recurrent neck; 3, recurrent aneurysm; 4, large saccular recurrences.

Macroscopic Photography and Pathology

Macroscopic and microscopic stereophotographs of cut sections of arteries and “en face” views of the neck of aneurysms were performed using a computerized imaging system (Clemex). The carotid wall was longitudinally opened to expose the luminal surface of the neck of aneurysms. Neointima formation and recanalization at the neck of aneurysms were evaluated according to a qualitative scoring system. (Raymond J. et al, Stroke, 2002; 33:421-427; Raymond J. eta l., Stroke, 2002; 33:850-855) Neointima formation was given a score of 0 when a thick neointima completely sealed the orifice; score of 1 when the neointima was similarly sealing the neck, but small areas of recanalization were seen between the neck and the wall of the aneurysm; score of 2 when a crescent of recanalization was present around the neointima covering the coil mass; score of 3 when recanalization affected the coil mass, the neointima covering being only partial; and score of 4 when no neointima, only thrombus, covered most of the coil mass. Arteries and aneurysms were studied after formalin fixation, axial sectioning, and staining with hematoxylin-phloxine-saffron (HPS) and Movat pentachrome stain. Immunohistochemistry served to characterize cells using antibodies to smooth muscle α-actin and factor VIII. (Raymond J. et al., AJNR Am J Neuroradiol. 2002; 23:1710-1716; Raymond J. et al., AJNR Am J Neuroradiol. 2002; 23:129-138) Sections of normal or denuded arteries harvested at the time of euthanization were immunostained for factor VIII for confirmation that the endothelium was effectively intact or denuded.

After each manipulation, the ANBD was inspected and rinsed in formalin, and for each animal the cytological material was pooled and collected by centrifugation. The resulting material was embedded in paraffin, sectioned and stained with HPS, and immunostained for α-actin, CD31, CD34, and factor VIII.

Statistics

Angiographic results of the arterial occlusion model were compared using a 2-sided X² test with continuity correction.

Independent sample t tests were performed to compare aneurismal diameters, volumes, and packing densities.

The evolution of angiographic scores of bifurcation aneurysms with time was analyzed using Wilcoxon signed rank tests whereas angiographic and neointimal scores of the 2 groups were compared using the Mann-Whitney test.

Results

Single-Coil Arterial Occlusion Model

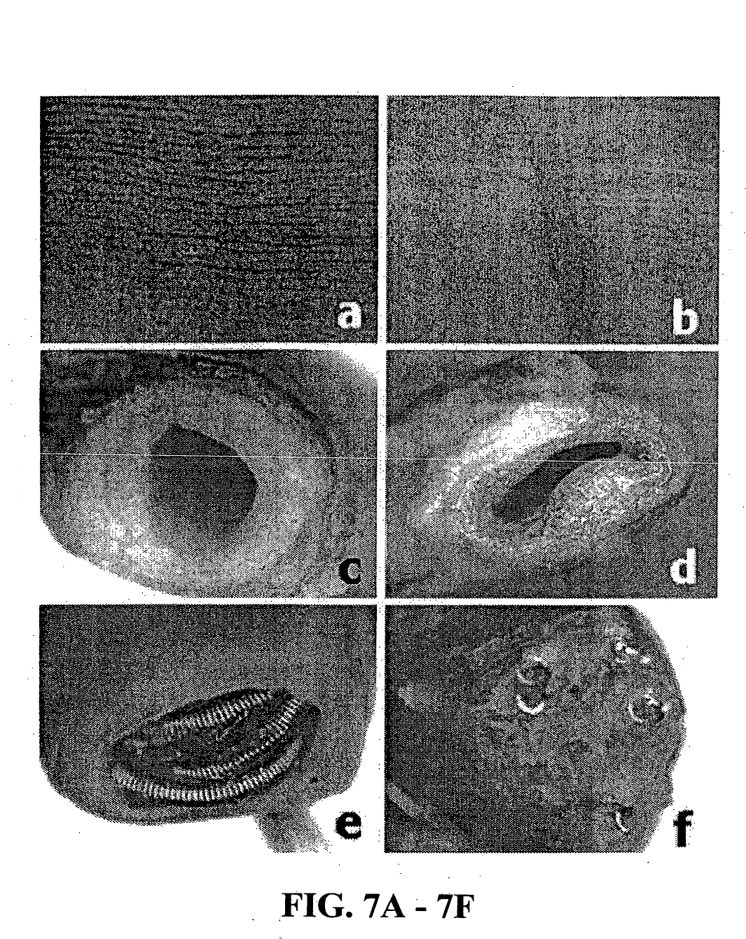

All arteries embolized with platinum coils recanalized (16/16), whereas most arteries (12/16 or 75%) denuded before coil embolization remained occluded at 3 and 12 weeks (P<0.001). At postmortem photography, arteries treated with coils only were recanalized, whereas most arteries treated by denudation and coiling were occluded, as shown in FIGS. 7e and 7f. Arteries that were denuded but not coiled, as well as intact arteries, remain patent at all times (FIGS. 7c and 7d).

Arteries that were immediately fixed for inspection after endothelial denudation showed inhomogeneous erosions of the intima, fading of normal endothelial ridges, and rare lacerations (FIG. 7b) . In some specimens studied by histopathology; the endothelial lining, as well as in rare places, the internal elastic lamina, were removed, presumably where lacerations occurred.

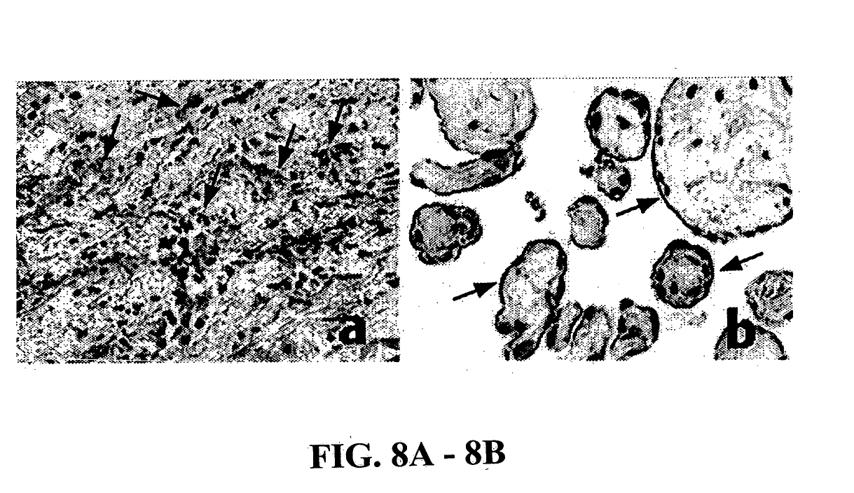

Arteries studied at 10 days after coil embolization showed early endothelial coverage of the clot and well-defined endothelialized channels, whereas they could not be found in arteries treated by denudation before coil embolization (FIG. 8a).

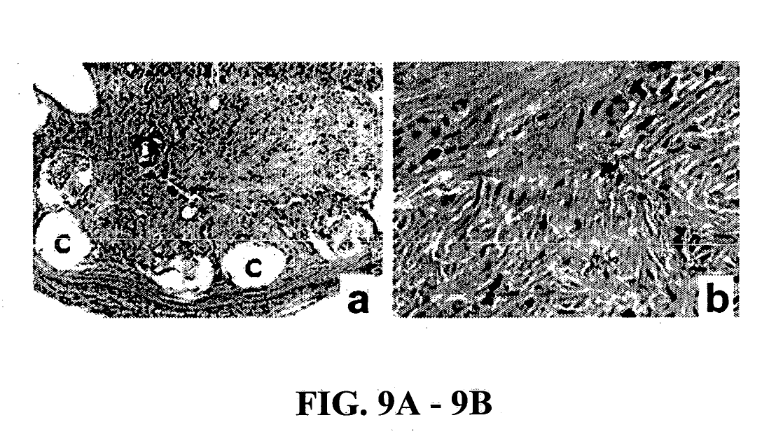

Arteries treated by coil embolization only showed partial filling with neointimal tissue, but constant recanalization (FIGS. 8a and 8b). Pseudopolypoid endothelialized structures were sometimes found within recanalized spaces (FIG. 8b). Arteries treated by denudation and coiling and studied at 3 months were filled with vascularized connective tissue, without endothelialized recanalizing spaces (FIGS. 9a and 9b). Arteries that were denuded but not coiled showed minimal patchy neointimal formation at 3 months. Material was recovered from the ANBD in ≈25% of animals. It consisted of fibrin, red blood cells, leukocytes, and rare parietal cells, some of which stained positive for factor VIII.

Bifurcation Aneurysm Model

Initial aneurysm dimensions, angiographic results, and neointimal scores are summarized in the Table 1.

There was no significant difference in diameters and neck width between the 2 groups, but aneurismal volumes tended to be larger in lesions treated by denudation, a difference that did not reach significance (P=0.069). The packing density (the coil/aneurysm volume ratio) was less in lesions treated by endothelial denudation (P=0.010).

The first animal died of carotid thrombosis that presumably started at the Y-shaped bifurcation that was inadvertently de-endothelialized by protrusion of the ANBD outside the aneurysm during manipulations. This complication could be prevented in the 7 animals with T-type bifurcations by assuring the intraaneurysmal position of the device. None of these 7 animals showed neurological deficits during follow-up.

TABLE 1

| | Mean | | | Mean | | | | | Median |
|---|---|---|---|---|---|---|---|---|---|
| | Diameter (mm) | | Mean Neck | Aneurysm | Mean | Median Angio- | | | Neointimal |
| | Long | Short | Width | Volume | Packing | graphic Scores | | | Scores |
| | Axis | Axis | (mm)* | (mm³)* | Density | T0 | T3weeks | T3months | T3months |
| Coil | 13.20 ± | 7.23 ± | 5.69 ± 1.17 | 369.13 ± 0.12 | 0.32 ± 0.12 | 2 | 3 | 4 | 2 |
| | 4.39 | 0.093 | | | | | | | |
| Coil + | 16.07 ± | 8.13 ± | 5.57 ± 0.092 | 576.33 ± 225.84 | 0.16 ± 0.06 | 2 | 1 | 1 | 1 |
| denuda | 2.28 | 1.16 | | | | | | | |
| tion | | | | | | | | | |
| P | 0.159† | 0.137† | 0.843† | 0.069† | 0.010† | 1‡ | 0.073‡ | 0.011‡ | 0.026‡ |

Aneurysms treated with coils without previous denudation tended to recur, with angiographic scores significantly worse at 12 weeks as compared with T₀(P=0.015). Lesions treated with endothelial denudation before coil embolization were stable. There was a significant difference between median angiographic scores of the 2 groups at 12 weeks (P=0.011).

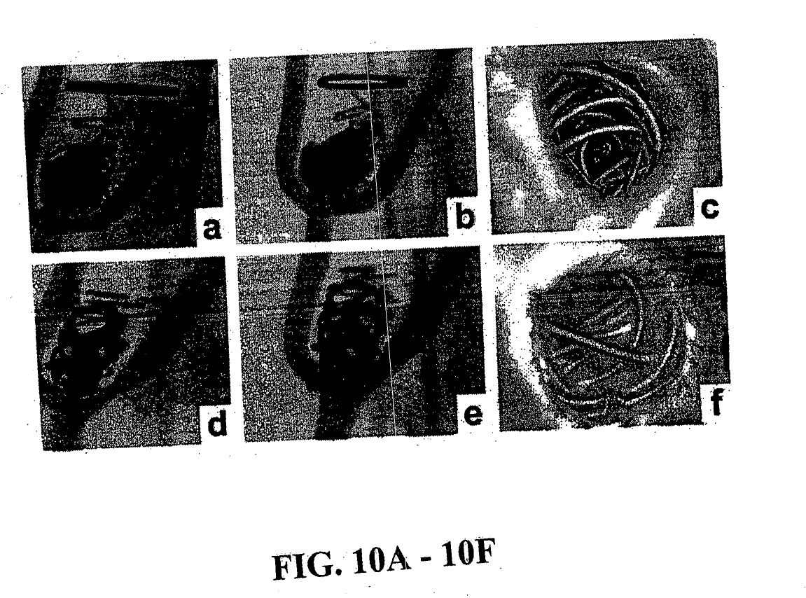

The median neointimal score of aneurysms treated with denudation before coil deposition was 1, a significant difference as compare with lesions treated with coils only (median score=2; P=0.026) (FIG. 10).

Discussion

We have previously shown that recanalization after coil occlusion of arteries is a cellular process that can be blocked by in situ beta radiation. (Raymond J. et al, Stroke 2002; 33:421-427; Raymond J. et al, Stroke 2003; 34:1262-1268) The process occurs within 2 weeks after occlusion but is only possible at this early stage. If the artery is occluded 2 to 3 weeks after embolization, it remains occluded thereafter for at least 3 months. (Raymond J. et al, Stroke 2002; 33:421-427; Raymond J. et al, Stroke 2003; 34:1262-1268) Because pathology at 3 months consistently reveals connective tissue replacement of the lumen in arteries that were not recanalized, we propose that his process can only occur within the provisional fibrin matrix that follows coil occlusion. In this model, once this fibrin matrix is replaced with a collagenous matrix, which occurs within the first weeks, recanalization is no longer possible, at least for 3 months. (Kroon ME et al, Angiogenesis 2002; 5:257-265)

Recanalization is associated with early endothelial invasion of the clot (FIG. 8a). (Raymond et al., Stroke 2003; 34:1262-1268) Circulating progenitor endothelial cells have been documented and their role in physiological or pathological angiogenesis is now recognized. (Iwaguro H. et al, Circulation 2002; 105:732-738) After denudation of the intima, they could have been a source of endothelial cells to repopulate the clot and promote recanalization. Although we cannot exclude a role for progenitor cells, failure of recanalization after endothelial denudation suggests that these cells cannot offer the spatial and temporal continuity necessary to effectively recanalize the lumen. One interpretation of our findings is that only cells from the original endothelial layer can migrate rapidly and form a continuous nonthrombogenic sheet to offer a connecting channel throughout the organizing clot.

The vascular wall alterations caused by the device have not been exactly defined in this study. Although the goal was to remove the endothelial lining, the resulting injury was not selective, nor have we proven that denudation was homogeneous and complete. The mechanical maneuvers may have caused deeper injuries, perhaps involving the internal elastic lamina, or medical trauma and secondary stimulation of neointima formation, as found in balloon injury models. However, the arterial occlusion model was previously tested with a combination of intraluminal stents and coils and, unless the devices were radioactive, occlusion was consistently followed by recanalization despite the trauma caused by the stent deployed by balloon inflation. (Raymond J. et al, Stroke 2003; 34:1262-1268)

Reduced blood flow and shear stress, which may be consequences of coil occlusion, augment endothelial cell proliferation and increase expression of platelet-derived growth factor (PDGF)-A and PDGF-B mRNA, but decrease nitric oxide synthase, responsible for production of nitric oxide, a prototype inhibitor signal from the endothelium that may be important in vessel wall quiescence. (Mondy J S et al., Circ Res. 1997; 81:320-327; Malek A M et al, Pediatr Neurosurg. 1997; 27:182-189) Thus, prevention of this mechanism by endothelial denudation may also explain how this procedure led to inhibition of recanalization.

Other mechanisms have to be considered such as differences in the composition of the initial fibrin-platelet clot when the coils were deployed in a denuded artery. Activated platelets trigger an inflammatory reaction with increased endothelial expression or secretion of uPA, tPA, UPAR, MT1-MMP, MCP-1, and ICAM-1, (May A E et al., Circulation 2002; 106:2111-2117; Gawaz et al., Circulation 1998; 98:1164-1171 and Dickfeld et al., Cardiovasc Res. 2001; 49:189-199) molecules that may favor recanalization or recruitment of cells involved in recanalization after coil occlusion. Denudation of the endothelial layer before coil embolization may prevent this chain of events, and thus favor collagen replacement of the fibrin clot with permanent occlusion of arteries.

We have chosen a T-shaped, rather than a Y-shaped, model because the configuration of the bifurcation permitted denudation at the neck with lesser risks of parent vessel complications. This model, which features smaller necks, also has a tendency to recur at 3 months after coil embolization, although to a lesser degree. (Raymond J. et al., AJNR Am J Neuroradiol 2002; 23:1710-1716; Turk A S et al., Stroke 2001; 32: 492-497; Raymond J. et al., Radiology 2001; 2001:318-326 and Raymond J. et al, AJNR Am J Neuroradiol. 2003; 24:11778-1784) Clinical recurrences may have multiple causes, but arterial recanalization at 2 weeks and angiographic recurrences observed within 3 months in canine are reproducible mechanisms that were effectively prevented by denudation.

There was a difference in aneurismal volumes that did not reach statistical significance, and a significant difference in packing densities (the ratio of total coil length on aneurismal volume) between the 2 groups. After mechanical denudation, occlusion of aneurysms occurred earlier after the introduction of a lesser length of platinum coils. We did not attempt to reach the same packing density, for fear of causing extrusion of clot and complications.

Conclusion

Endothelial denudation before coil deposition can prevent recanalization after coil embolization of canine arteries. Angiographic scores were significantly improved 3 months after endovascular treatment of experimental bifurcation aneurysms.

1. A device for denuding the endothelium of a vascular cavity wall, the device comprising:
an endovascular tool for performing an endovascular treatment of the vascular cavity; and
at least one denuding element adapted to remove at least part of the endothelium of the vascular cavity wall while avoiding wall perforation, the at least one denuding element being attached to the endovascular tool;
whereby the denuding of the endothelium of the vascular cavity wall maximizes thrombosis and prevents recanalization in the vascular cavity.

2. The device of claim 1, wherein the at least one denuding element is removably attached to the endovascular tool.

3. The device of claim 1 wherein the at least one denuding element removes the at least part of the endothelium by a mechanical scraping action.

4. The device of claim 1, wherein the at least one denuding element is a chemical agent adapted to remove the at least part of the endothelium while avoiding wall perforation.

5. The device of claim 1, wherein the at least one denuding element removes the at least part of the endothelium through the use of at least one of heat, cold, radiofrequency, laser and electricity.

6. The device of claim 1, wherein the endovascular tool is a guidewire and the at least one denuding element is attached to a tip of the guidewire.

7. The device of claim 1, wherein the endovascular tool is a microcatheter and the at least one denuding element is attached to a tip of the microcatheter.

8. The device of claim 1, wherein the endovascular tool is a microballoon and the at least one denuding element is attached to an external surface of the microballoon.

9. The device of claim 1, wherein the endovascular tool is a coil and the at least one denuding element is attached to an external surface of the coil.

10. A method of preparing a vascular cavity having blood therein before vascular cavity occlusion, the method comprising the steps of:
a) endovascularly disposing at least one denuding element inside the cavity; and
b) using the at least one denuding element to remove at least part of an endothelium of a cavity wall while avoiding wall perforation;
whereby the removal of at least part of the endothelium of the cavity wall maximizes thrombosis and prevents recanalization in the vascular cavity after vascular cavity occlusion.

11. The method of claim 10, wherein before step a) the method further comprises a step of endovascularly disposing a microcatheter inside the cavity, and step a) further comprises inserting the at least one denuding element through a lumen of the microcatheter in order to dispose the denuding element inside the cavity.

12. The method of claim 10, wherein after step b) the method further comprises a step of removing the denuding element from the vascular cavity.

13. The method of claim 10, wherein the at least one denuding element is attached to an endovascular tool.

14. The method of claim 10, wherein the at least one denuding element removes the at least part of the endothelium by a mechanical scraping action.

15. The method of claim 10, wherein the at least one denuding element is a chemical agent adapted to remove the at least part of the endothelium while avoiding wall perforation.

16. The method of claim 10, wherein the at least one denuding element removes the at least part of the endothelium through the use of at least one of heat, cold, radiofrequency, laser and electricity.

17. The method of claim 13, wherein the endovascular tool is a guidewire and the at least one denuding element is attached to a tip of the guidewire.

18. The method of claim 13, wherein the endovascular tool is a microcatheter and the at least one denuding element is attached to a tip of the microcatheter.

19. The method of claim 13, wherein the endovascular tool is a microballoon and the at least one denuding element is attached to an external surface of the microballoon.

20. The method of claim 13, wherein the endovascular tool is a coil and the at least one denuding element is attached to an external surface of the coil.
